(19) United States
(12) Patent Application Publication
(10) Pub. No.: US 2022/0354567 A1
(43) Pub. Date: Nov. 10, 2022

(54) ELECTRODE ARRANGEMENT IN A CARDIAC ABLATION CATHETER AND METHODS FOR USE

(71) Applicant: Focused Therapeutics, Inc., Salt Lake City, UT (US)

(72) Inventors: Yonathan F. Melman, Salt Lake City, UT (US); Paul MELMAN, Newton, MA (US); Meir M. BROSH, Newton Center, MA (US)

(21) Appl. No.: 17/722,533

(22) Filed: Apr. 18, 2022

Related U.S. Application Data
(60) Provisional application No. 63/183,181, filed on May 3, 2021.

(51) Int. Cl. A61B 18/14; A61B 18/12

(52) U.S. Cl. CPC A61B 18/1492; A61B 18/1206; A61B 2018/00577

(57) ABSTRACT

A novel cardiac ablation catheter is based on the principle that the field gradient near the electrode surface is reduced by a truncated dome shape which reduces the ratio of the current magnitude near the electrode-tissue interface to that in the tissue. Thus, for a given power, a deeper lesion can be created at a lower applied power reducing the risk of steam pop and overheating of the surrounding blood pool. The result is a reduction in spurious current which does not contribute to tissue ablation but undesirably increases heating of the blood pool near the ablation site. Methods of use are also described.

### CROSS-REFERENCE TO RELATED APPLICATIONS

This application claims priority to U.S. Provisional Patent Application No. 63/183,181 filed on May 3, 2021.

### BACKGROUND

### Field

The present disclosure relates generally to minimally invasive devices and methods for delivering electrical energy (RF or pulsed field) energy to tissue. Aspects of the disclosure related to configurations of ablation catheters, and more specifically to electrodes adapted to use in RF or pulsed field delivery catheters. Aspects of the disclosure pertain to devices and methods for treating cardiac arrhythmias using electrical energy delivery catheters using electrode configurations described herein.

### Background

Radiofrequency catheter ablation (RFCA) is a cornerstone of minimally invasive electrophysiology in the treatment of cardiac arrhythmias. The technique involves the application of a high-frequency alternating current to the cardiac tissue (e.g., myocardium) via one or more electrodes disposed on an access catheter. Electrodes are designed to develop a high current density that leads to tissue heating and destruction (i.e., ablation) in a highly controlled manner. RF ablation techniques are used to treat virtually every cardiac arrhythmia. A frequent limitation of standard RF ablation approaches is the difficulty to ablate tissue that is remote from an endocardial or epicardial surface. This is especially true for substrates arising in the intraventricular septal or other intraventricular myocardial locations.

The depth to which ablation can be carried out is limited by heating of both the cardiac tissue and the blood pool itself. Currently, irrigated tip monopolar ablation catheters are limited in their ability to ablate tissue beyond depths of approximately 6-9 mm. Due to factors such as the cylindrical geometry of the prior-art catheters and electrodes, the electric field and current density generated have the highest magnitude near the catheter-tissue interface and the vast majority of energy during RF delivery is dissipated in the first few millimeters of tissue. This dissipation results in a rapid fall off in tissue heating. Thus, the depth of ablation is limited because once the current density exceeds a certain threshold, excessive tissue heating occurs. While raising power output from the RF generator increases current delivery, doing so may increase the risk of explosive decompression (a so-called “steam pop”) due to excessive tissue heating and creation of a steam pocket within the tissue, as well as char formation due to coagulation of the blood pool. Steam pops are dangerous and have the potential to result in severe complications. When the excessive heating occurs in the blood pool, a coagulum can form on the catheter surface. The coagulum, if dislodged from the catheter can embolize and block cerebral blood vessels, leading to embolic stroke.

Other RF approaches such as bipolar ablation, half-normal saline (HNS) irrigation, and needle-tip catheters have been introduced to overcome limitations in the procedure inherent in conventional RF catheter designs, but these techniques have their own disadvantages and challenges.

All arrhythmias are caused by the abnormal function of electrically excitable cardiac tissue. Arrhythmias can be caused by either conduction disturbances (e.g., abnormal rapid firing) abnormal impulse formation (i.e., focal arrhythmias), or by the formation of loops of electrical activation that propagate back on themselves, thereby perpetuating themselves (i.e., reentrant arrhythmias).

The technique of RFCA involves introducing a catheter into the heart via the arteries, veins or directly into the pericardial membrane which surrounds the heart. Depending upon the specific arrhythmia, the catheter may be introduced into any of the heart's four chambers' interior or exterior. A variety of established techniques are then used to analyze the arrhythmia and identify areas of the heart that are critical to propagating or maintaining it. Once such an area is determined, a specific site is identified, and ablation is performed. Ablation involves either cooling the tissue (called cryo-ablation) or more commonly heating the tissue with the application of high-frequency (or radio frequency) alternating current (AC). The ablation of the tissue destroys the specific part of the heart responsible for or crucial to the arrhythmia. Cardiac RFCA has become a widely used and practiced technique. In the modern era, success rates for treating arrhythmias are as high as 95-98%, with low complication rates for what is often an outpatient procedure.

The conventional technique of RFCA involves placing an electrode-tipped catheter in contact with the critical area inside the heart. Electrical current is passed between the catheter tip and a large grounding pad placed in electrical contact with the patient's skin. The high current density generated at the catheter tip leads to heating of the tissue and an irreversible destruction if maintained at a sufficient duration and intensity. A disadvantage of the conventional technique is that an arrhythmia target may not be on an accessible surface of the heart permitting direct access by an RFCA catheter, making the effective and accurate delivery of energy problematic. The heart contains muscle that may be over a centimeter in thickness and arrhythmia circuits or targets may lie deep in the muscle. Therefore, there exists the need for improved devices and methods for delivering RF energy deeper into the muscle.

RF ablation is limited in its ability to deliver deep lesions, as most of the energy delivered to the tissue is dissipated in the first few millimeters from the catheter tip. Conventional RFCA catheters are also limited because the depth of an RFCA ablation lesion is ultimately constrained by the ability to maintain a sufficient current density at a given distance from the catheter. RF catheters today have a cylindrical tip. As RF energy output is increased in any attempt to ablate deeper into the tissue, the current density in proximity to the catheter tip continues to rise, ultimately reaching a point where water in the cardiac tissue or blood starts to boil. This can result in the buildup of pressure and subsequent explosive, often audible, decompression, referred to as a “steam pop.” This can lead to perforation of the heart's chambers or damage to cardiac structures, which in the worst-case scenario is a life-threatening emergency. Cylindrically symmetric RF catheters also deliver RF energy into the blood which they are in contact with during ablation. Excessive heating of the blood leads to a denaturation of blood proteins and destruction of red blood cells, leading to a coating of the catheter, termed “char”. This char can impede energy delivery and thus further ablation, and more seriously can break free of the catheter as a solid mass, possibly causing an embolic stroke by occluding small blood vessels in the brain. Various improvements in the field of RF ablation have included the introduction of various types of irrigated-tip catheters which mitigate excessive heating and char formation at the electrode surface by circulating saline solution through the tip itself during ablation.

### SUMMARY

The disclosed apparatus and methods include a novel ablation catheter. Focused Electric Field (FEF) is a novel technology which can be employed to ablate much deeper than conventional RF catheters. FEF ablation is based on a principle of electrostatics that the electric field of a charged conductive surface will be highest at areas of small radius of curvature. Unlike traditional RF ablation, FEF ablation uses a catheter tip electrode that has a novel at least partial dome shape at the catheter tip and dielectric insulation along the sides of the catheter and on its rim. Current is delivered from a conductive surface at the catheter tip. This design reduces the electric field at the catheter surface and leads to a markedly different electric field distribution with a much slower fall-off of the field with distance from the electrode (and hence of resistive heating). This more uniform electric field allows for deeper ablation without the risk of excessive tissue heating and steam pops during energy delivery. Here is presented ex vivo data demonstrating the FEF catheter's ability to ablate significantly deeper than current irrigated-tip catheters. According to embodiments of the present disclosure, novel catheters can be used to deliver either radiofrequency (RF) energy or to perform pulsed-field ablation (PFA). Other uses or modalities consistent with the disclosure are also possible.

Whether FEF technology is used in conjunction with RF energy or with emerging technologies such as pulse-field ablation (PFA) or electroporation, the ability to deliver electrical energy deeper into the tissue while avoiding superficial tissue heating and energy loss is essential to ablating arrhythmogenic foci at mid-myocardial or ventricular intramural sites.

To the inventors' knowledge this disclosure includes examples which are the first proof of concept of FEF technology. According to some aspects of the disclosure, ablation can be performed in a perpendicular, or vertical, direction with respect to the tip axis as well as in the parallel direction with respect to the tip axis. According to aspects of the present disclosure, the catheter can produce a more uniform electric field near the electrode surface. This tip geometry reduces the ratio of the current magnitude near the electrode-tissue interface to that in the tissue. Thus, for a given power, a deeper lesion can be created at a lower applied power reducing the risk of steam pop and overheating of the surrounding blood pool. According to illustrative embodiments disclosed herein, the electrode tip can be configured to have an at least partial dome shape, and in still further illustrative embodiments the at least partial dome shape can be configured as at least partially toroidal up to and including entirely toroidal.

In one illustrative embodiment of the disclosure, an ablation catheter for ablating cardiac tissue has a catheter body with a longitudinal axis and a distal tip and an ablation electrode proximate to the distal tip of the catheter body, wherein the electrode has an at least partial dome shape which is at least partially electrically conductive. In some exemplary embodiments, the ablation catheter is configured to ablate tissues other than cardiac, for example nerve tissue fluid for treatment of pain, tumor tissues and dorsal penile nerves for the treatment of premature ejaculation.

The ablation catheter in some embodiments of the disclosure can have an electrode with a non-conductive ceiling region.

In still other embodiments of the disclosure, the dome shape of the electrode has a central axis which is parallel or substantially parallel to the longitudinal axis of the catheter body. In some embodiments, the central axis is perpendicular or substantially perpendicular to the longitudinal axis of the catheter body. In some embodiments, the central axis is at an angle other between perpendicular and parallel.

In other embodiments of the disclosure, an ablation catheter can comprise a plurality of electrodes spaced about the catheter body. In further embodiments, each of the plurality of electrodes have a central axis. In still further embodiments, one or more of the central axes are parallel to one another or can be at an angle to each other or in other embodiments one or more of the central axes are non-parallel to one another.

In yet other embodiments of the present disclosure, an ablation catheter can have a temperature sensor in proximity to the electrode. In other embodiments of the disclosure an ablation catheter includes at least one fluid aperture, which in various embodiments can include one or more of a fluid flow inlet aperture and a fluid flow outlet aperture. In various embodiments, the apertures can be used to deliver or aspirate liquid or gas. A fluid can help flush the target area and also provide a means for thermal management of the catheter tip.

According to some embodiments, an ablation catheter includes an electrode made of conductive mesh. In other illustrative embodiments the electrode is made of conductive foldable mesh.

In some illustrative embodiments of the disclosure, the electrode has a dome shape having a rim, and wherein the rim is covered by an insulating material.

In still further embodiments of the disclosure an ablation catheter system for ablating cardiac tissue comprising includes an ablation catheter having a catheter body having a longitudinal axis, the catheter body including a distal tip; at least one ablation electrode proximate to the distal tip of the catheter body; wherein the electrode has an at least partial dome shape which is at least partially electrically conductive; and an RF or PFA power generator connectable to the ablation catheter. In further embodiments the RF or PFA power generator is configurable to deliver RF or PFA power with time-dependent amplitude.

In yet another exemplary embodiment of the present disclosure, a method for cardiac ablation includes the steps of: a) providing an ablation catheter comprising a catheter body having a longitudinal axis, the catheter body including a distal tip and at least one ablation electrode proximate to the distal tip of the catheter body wherein the electrode has an at least partial dome shape which is at least partially electrically conductive; b) advancing the catheter tip to a cardiac treatment site of a patient; c) delivering RF or PFA power to the electrode of the ablation catheter to ablate cardiac tissue. In illustrative embodiments, the cardiac tissue proximate to the tip of the catheter during the delivering step is heated less than compared to an electrode not having a an at least partial dome shape. In other illustrative embodiments, the cardiac tissue proximate to the tip of the catheter during the delivering step exhibits fewer steam pops than compared to an electrode not having an at least partial dome shape. According to one illustrative method of the present disclosure, the cardiac tissue ablated is located greater than 0.6 cm from the catheter tip and is ablated more quickly than compared to an electrode not having an at least partial dome shape.

### BRIEF DESCRIPTION OF DRAWING(S)

The drawings are not necessarily to scale or exhaustive. Instead, emphasis is generally placed upon illustrating the principles of the inventions described herein. The accompanying drawings, which are incorporated in and constitute a part of this specification, illustrate several embodiments consistent with the disclosure and together with the description, serve to explain the principles of the disclosure. The patent or application file contains at least one drawing executed in color. Copies of this patent or patent application publication with color drawing(s) will be provide by the Office upon request and payment of the necessary fee. In the drawings:

FIG. 1 depicts a catheter assembly in accordance with disclosed embodiments.

FIGS. 2A-2B depict side and end views, respectively, of an exemplary perpendicular ablation catheter tip in accordance with disclosed embodiments.

FIGS. 3A-3B depict an exemplary parallel ablation catheter tip in accordance with disclosed embodiments in aligned side and end views.

FIG. 4A-4B depict an exemplary shape of an electrically conducting dome and a schematic depiction illustrating parameters used to model catheter electrode tips.

FIGS. 5A-5C are schematic representations, respectively, of a prior art ablation catheter tip, a control catheter tip and an illustrative catheter tip according to the present disclosure.

FIG. 6 is graph showing relative power density by tissue depth of illustrative embodiments of the present disclosure compared to the prior art.

FIG. 7 shows TTC stained tissue sections post ablation comparing prior art and catheters according to aspects of the present disclosure.

FIG. 8 shows real-time thermal imaging comparing development of lesions using normal saline (NS) and half-normal saline (HNS) irrigation fluid in prior art catheters and illustrative catheters according to aspects of the present disclosure.

FIG. 9 is a graph summarizing the variation of temperature with depths from catheter-tissue interfaces.

FIG. 10 is a graph showing planimetered area of 100° C. isotherm during ablation.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

According to exemplary embodiments of the disclosure, various geometries of the indentation at the tip can reduce spurious currents which do not contribute to tissue ablation but increase heating of the blood pool. According to illustrative embodiments, a method is described to modulate the applied power to further increase of the ablation depth.

According to further aspects of the disclosure the use of a shaped catheter tip designed to reduce the electric field magnitude at the catheter-tissue interface is described. The electrical field generated by this tip falls off more slowly as the distance from the electrode surface into the tissue increases. This increases the magnitude of the field penetrating the tissue relative to its value at the electrode interface and thus extends the depth of safe ablation without causing high field values near the tip itself. The geometry of the shaped catheter tip is such that an indented surface of the catheter tip faces the surface of the cardiac tissue and can reduce the magnitude of the electric field at the catheter tissue interface.

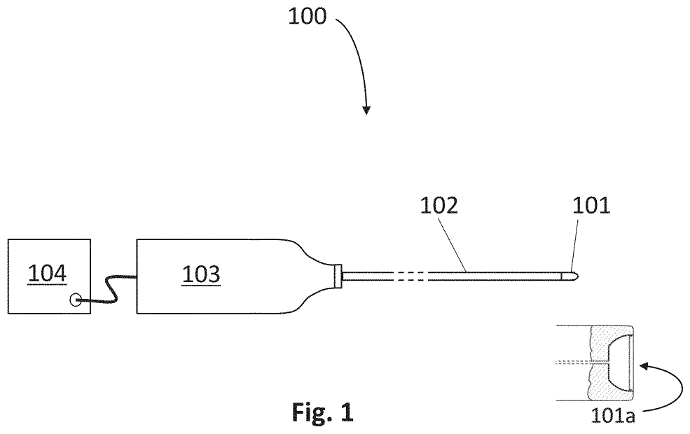

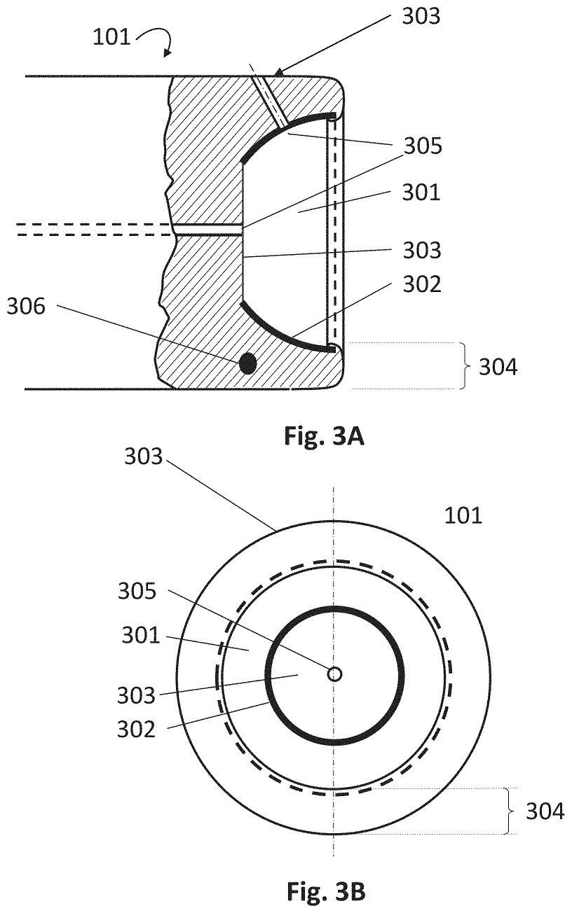

Referring to FIG. 1, a schematic view of an illustrative catheter assembly 100 is depicted. The tip 101 is at the distal end of catheter shaft 102. In alternative embodiments the tip 101 can be adapted as an electrode for placement on other locations on the catheter shaft or on other structures known in the art for accessing ablation sites within or on the body. A magnified schematic view of an exemplary tip 101a is shown in the illustrative schematic depiction, which is described in greater detail in FIGS. 3A and 3B below. A handle 103 is provided for manipulation of the catheter shaft, but in alternative embodiments the handle may not be necessary, for example during robotically assisted procedures. An RF or PFA power generator 104 can be provided to supply power to the tip, which delivers ablation energy to a delivery site. The generator can in some embodiments provide RF or PFA power with time-dependent amplitude.

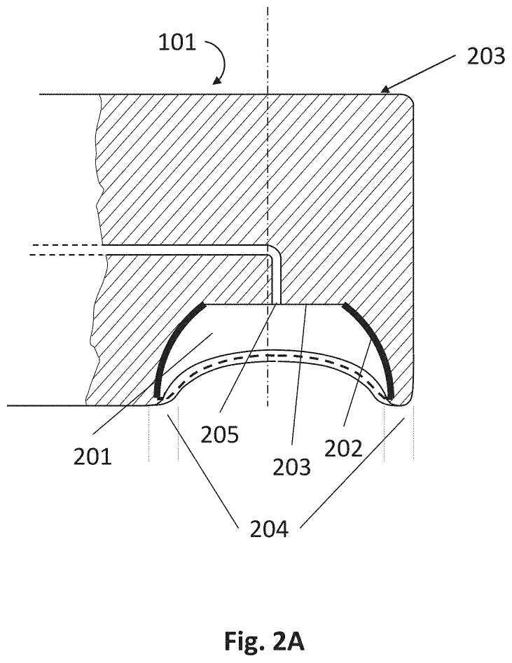

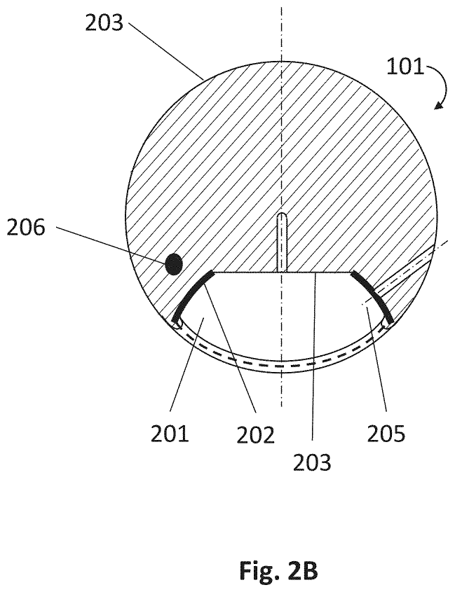

Referring to FIGS. 2A and 2B, a further illustrative embodiment of the catheter tip 101 is depicted. In the illustrative embodiment the tip can be configured to ablate tissue located lateral or perpendicularly with reference to the longitudinal axis of the catheter, where the central axis of the dome is perpendicular to the major axis of the catheter body or tip. As shown in FIG. 2B the electrode tip 101 is shown in cross-section and comprises a conductive at least partial dome shape 201 of the indented cavity 202 and non-conducting surface 203. A catheter may have a plurality of at least partial dome shaped electrodes spaced along the length of the catheter, with the respective dome axes aligned, for example parallel or with the axes lying in parallel planes, or with the axes lying in planes non-parallel to one another. The electrodes can reside in a single tip structure, or in separate bodies. As shown, the top central area, or ceiling, of the dome can be non-conductive. The exterior surfaces of the tip 101 can also be non-conductive. In some embodiments the surface 203 can be electrically insulated, thermally insulated or both. Surface 203 does not need to have any specific shape such as that depicted in the illustrative embodiment. As shown in FIG. 2A, the indentation can be further defined by a rim 204 of the tip having a radius of curvature or chamfer, such as for example a bead, a fillet or a bevel. In some embodiments the rim 204 can be electrically insulated, thermally insulated or both. FIG. 2A represents a side view in partial cross-section, respectively, and further depicts a flow port 205 for introduction of irrigation or coolant fluid. One or more flow ports can be provided for either delivering or aspirating fluid. One or more temperature sensors 206, as schematically represented, can be provided in various embodiments. The temperature sensor can take the form of a thermistor, a thermocouple or other type of sensor. In use, the electrode is not heated directly by the delivered RF energy, but by conduction from contact with heated tissue. The temperature sensor can be mounted in proximity to the electrode. The temperature sensor can therefore help to provide a proxy for the temperature of the tissue being ablated to help prevent overheating of the tissue. As mentioned previously, overheating of the electrode can lead to degraded performance and the risk of embolic stroke. A temperature measured below a certain threshold may also be an indication of poor or incomplete contact between the ablation electrode and the tissue, resulting in low heat conduction from the tissue to the tip.

FIGS. 3A and 3B depict a further illustrative embodiment of the catheter tip 101 according to the present disclosure. The figures present, respectively, a side-view and front view of the tip 101. The depicted embodiment can be employed to deliver ablation energy distally from the distal end of the electrode tip, where the central axis of the dome 301 is parallel to or coincident with the major longitudinal axis of the catheter or tip. The axis of the dome does not need to be exactly parallel or coincident but may be inclined or declined depending on intended use. As shown, the tip 101 comprises an at least partial dome-shaped cavity 301 forming a conductive cavity 302 and a non-conducting surface 303 that can be of any expedient shape. In some embodiments the surface 303 can be electrically insulated, thermally insulated or both. The cavity can be partially defined by a rim 304 having a radius of curvature or chamfer, such as for example a bead, a fillet or a bevel. In some embodiments the rim 304 can be electrically insulated, thermally insulated or both. The tip can also comprise one or more flow ports 305 for delivering or aspirating fluid, for example for irrigation or cooling. A temperature sensor 306, as schematically represented, can be provided proximate to the electrode.

In some embodiments, the electrode can be made of a conductive mesh. In further embodiments, the conductive mesh can be foldable. In other embodiments the electrode can be formed of a conductive metallic material.

### Examples

An FEF catheter was constructed with a hemispherical truncated dome forming an electrode tip as described herein. Ablation was performed ex vivo in porcine hearts and ablation characteristics were examined using both tissue sectioning and real-time thermal imaging.

As described below in detail, a THERMOCOOL SMARTTOUCH SF porous-tip catheter available from Biosense Webster was used for comparison. RF lesions were 9.1±1.0 mm wide by 6.1±1.1 mm deep with ablation using an irrigated-tip Thermocool SF catheter. In contrast, lesions created using FEF ablation were 16.1±2 mm wide and 15.2±1.1 mm deep. Steam pops were much less frequent with FEF technology (6.7% incidence for FEF vs. 15% with Thermocool). Thermal imaging demonstrated that in contrast to an irrigated tip RF catheter, the FEF catheter generated a uniform temperature profile down to a maximum depth exceeding 15 mm. As summarized in the table, a control catheter without the FEF tip but similarly insulated to the FEF tip did not ablate significantly deeper than the Thermocool catheter, confirming the role of the truncated dome in the FEF effect.

Biophysical Model of FEF Ablation and Methodology

Described herein is the electric field model used to demonstrate that FEF catheters constructed in accordance with disclosed embodiments achieve a more uniform field distribution. Electric field along the axis of symmetry of the catheter tip are computed for an FEF-generated field and that of a standard tip for comparison.

The field along the symmetry axis is calculated as follows. The electric field of a conductive structure can be calculated using Coulombs law which relates its value E (field amplitude) to a point charge Q at a distance r, where the field E is a vector pointing along r with magnitude:

$$E = \frac{2 π}{{ε}_{o}} \frac{Q}{{r}^{2}} .$$

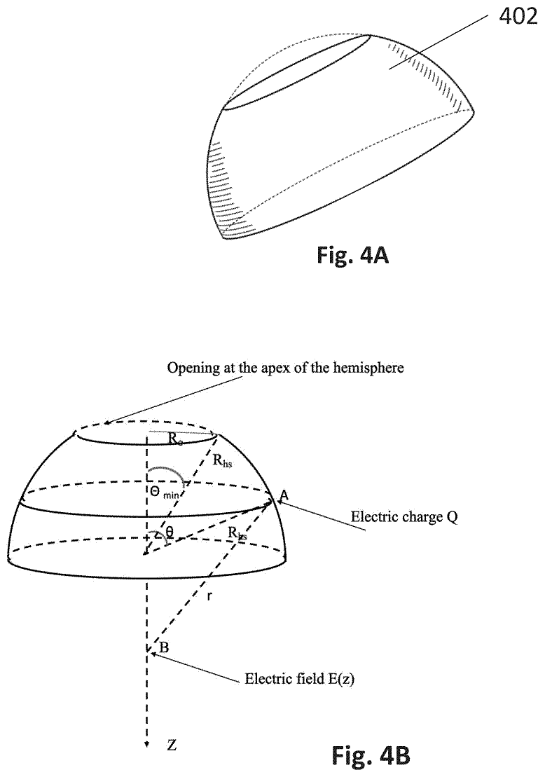

FIG. 4A depicts an illustrative shape of a conducting at least partial dome shape according to one aspect of the disclosure. It is to be appreciated that the dome shape needn't be described by a spherical section, but could be ellipsoid, ovoid or other curved reflective forms. It is also to be appreciated that the curved surfaces can be approximated by flat surfaces, e.g. a geodesic dome. The model catheter tip with relevant parameters used in our calculations is shown in FIG. 4B. The total field due to a complex shape such as a catheter tip at any point can be obtained by integrating the charge over the total area of the structure. The electric field formed by a hemispherical or toroidal catheter tip can be calculated according to this integration, and the model also accounts for an opening at the apex of the hemisphere or toroid to allow communication with the saline bath. The opening is circular, and it affects the distribution of the electric field.

With reference to FIG. 4B, Here Rₕₛ is the radius of the hemisphere; Rₒ is the radius of the opening, A is the location of the differential charge dQ=σdA where σ is charge density and dA a differential of the surface area. B is the point where the electric field contributed by the charge at A is computed, θ is the angle between the symmetry axis of the hemisphere and the point A. θmin is the angle subtended by the opening. To compute the total field at B, integration over the whole surface of the hemisphere excluding the area of the opening is performed.

$$\begin{matrix}E ( z ) = 2 π k σ * {\int }_{π / 2}^{θ min} \frac{2 π {R}^{2} * ( z + R * cos θ ) * sin θ * d θ}{{( {R}^{2} + {z}^{2} + 2 * R * z * cos θ )}^{3 / 2}} = \\ 2 π k σ * ( \frac{R + z * cos θmin}{{z}^{2} * \sqrt{{R}^{2} + {z}^{2} + 2 * R * z * cos θmin}} - \frac{R}{{z}^{2} * \sqrt{{R}^{2} + {z}^{2}}} )\end{matrix}$$

Where

$$k = \frac{1}{4 πε}$$

and ε is the dielectric constant of the medium.

The field E(z), because of symmetry of the hemisphere, has only one component at point B, which is along the z direction. Its value depends on the distance from the hemisphere and the size of the aperture, θmin.

A standard electrode has a cylindrical tip and the shape of the tip when ablating end-on is a flat disc. The field of a flat disc at distance z from its center on its symmetry axis is given by:

$$E ( z ) = 2 π k σ * ( 1 - \frac{z}{\sqrt{{R}^{2} + {z}^{2}}} )$$

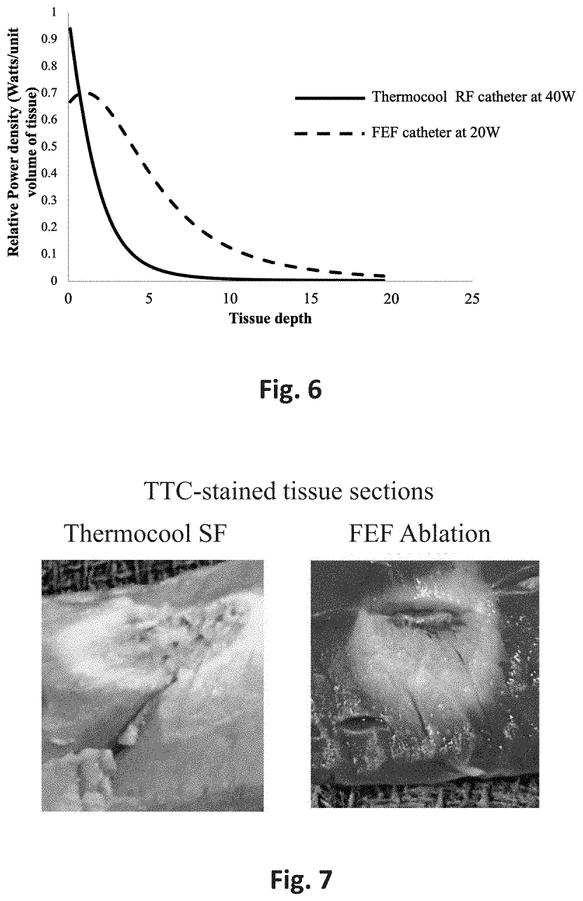

Squaring the two equations above for the hemispherical and commercial catheter tips yields an expression for the predicted power delivered to the tissue along the axis of the catheter during ablation. These data are graphed for comparison in FIG. 6, which compares the relative power density delivered from a Thermocool irrigated state-of-the-art catheter at a 40 W nominal setting to the FEF catheter at 20 W, due to the smaller surface area of the FEF compared to the Thermocool (34 mm² versus 14.4 mm²), in order to compare ablation conditions under similar surface current densities which would reflect their real-world use.

Catheter Construction

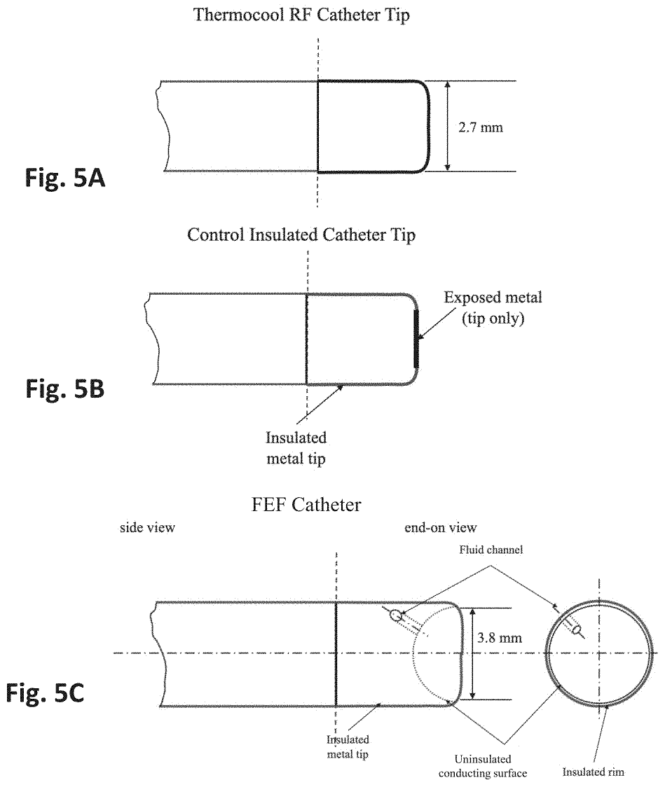

The FEF electrode for this study was constructed as a tip attached to a standard, commercially available electrode. The tip is machined from a copper cylinder, 5 mm in diameter and 8 mm in length. The tissue end of the rod is shaped so as to create a hollow depression. In the experimental catheter, the diameter of the hollow was approximately 3.8 mm. A 1 mm hole at the end of the catheter allows for communication of saline in the catheter tip with the circulating bath. The schematics of the FEF tip is shown in FIG. 5C. FIG. 5A shows schematics of a current-art RF catheter tip. The FEF catheter has an indented toroidal tip, and dielectric insulation around the sides. Ablation takes place with the distal tip of each catheter in an end-on orientation. FIG. 6 is a graph depicting the theoretical results of the biophysical model described in this disclosure, calculated as detailed herein, comparing the expected power delivered to the tissue at various depths for both the FEF and the commercial RF irrigated catheter calculated using the above described model. The graph depicts the calculation of the power delivered by the FEF catheter versus the Thermocool RF catheter along the long axis of the catheter. As shown; the power falls off much more slowly with distance in the FEF tip compared to a traditional cylindrical orientation of the Thermocool catheter. Due to the lower exposed surface area of the FEF catheter, the power delivered by the FEF catheter at 20 W is compared to the Thermocool at 40 W to arrive at a similar current density at the two ablating surfaces. Comparing the curves in FIG. 6, the RF catheter delivers much higher power density to the area immediately subjacent to the catheter, but the delivered power falls off rapidly with depth. The power delivered to the tissue falls to one-half the maximal value by 2.9 mm of depth. In contrast the electric field of the FEF catheter displays a very different behavior; the field at the catheter tip is lower initially and increases to a peak at a depth of 3.2 mm below the tissue surface. After reaching this maximum, the energy falls off with depth at a much slower rate than that of an RF electrode, falling to one-half of the maximal energy delivery at a depth of 7.0 mm.

The proximal end of the tip as used in these examples was attached to the end of a commercial tip in a way as to provide mechanically stable connection and ensure electrical contact between the two tips. The outsides of both tips were coated with an electrically insulating lacquer coating, and a plastic rim was applied around the FEF indented surface to complete the insulation. This prevents current from flowing directly into the surrounding blood pool and eliminates areas of sharp curvature where the electric field is highest. Hence, in the tip used in this experiment, the current flow was confined to the truncated surface.

As a further control, a catheter was constructed and insulated in an identical manner to the FEF catheter, but with a planar end instead of the toroidal truncated dome of the FEF surface. The same type of insulating material was used on the control insulated catheter tip and the FEF catheter, leaving only the tips exposed for ablation. A schematic of this tip is shown in FIG. 5B.

Experimental Tissue Preparation

Fresh intact porcine hearts were acquired within 2-4 hours of sacrifice. The LVs were harvested, and rectangular strips of myocardium were (2.5×5 cm×myocardial thickness [>2.0 cm]) excised. The LV myocardial samples were submerged in a circulating bath of NS (0.9% NaCl) continuously heated to 37° C. The ablation system for the FEF RF arm included: a RF3000 RF generator and a power-controlled FEF RF catheter. The ablation system for the irrigated RF catheter arm consisted of a Stockert RF generator, Smart Ablate irrigation pump (with flow rate set to 15/mi min during RF), and 3.5 mm THERMOCOOL SMARTTOUCH/Surround flow open irrigated-tip ablation catheter (Biosense Webster, Diamond Bar, Calif.). An indifferent electrode (grounding patch) in the fluid bath completed the electric circuit.

Radiofrequency Lesion Formation

RF lesions were created with the FEF ablation catheter's tip submerged in the fluid bath and positioned perpendicular to the endocardial surface of myocardial tissue samples during lesion applications. RF energy was delivered in a power-controlled mode. In initial experiments a significantly increased incidence of steam pops above 20 W was noted, while effective lesion formation was observable at this power output. Hence, for purposes of these examples, comparing ablation lesions to irrigated-tip Thermocool catheters, ablation applications were performed at 20 W. Slow and continuous lesion expansion over several minutes was observed. Ablation lesions were thus performed for either 2 minutes or 4 minutes per lesion. Tip temperature, starting impedance, and impedance drop were continuously monitored during each ablation lesion.

Irrigated Ablation Group

RF lesions in the irrigated catheter ablation group were performed with the ablation catheter tip submerged in the fluid bath and positioned perpendicular to the endocardial surface of tissue samples. Both NS (0.9% NaCl) or HNS (0.45% NaCl) were used for irrigation to allow comparison to RF ablation under a range of well-studied conditions. During RF applications, the catheter tip was irrigated with room temperature NS or HNS at a flow rate of 15 mL/min.

In both the NS and HNS groups, RF energy was delivered for 90 seconds per lesion at a constant power setting of 40 Watts (W), contact force of 10-15 grams (g). These settings were selected to produce optimal lesion size with little increase in lesion size with longer durations. Longer duration lesions were attempted for a more direct comparison to FEF ablation, however longer duration lesions at 40 W caused a high rate of steam pops so power was reduced to 30 W with NS irrigant for two and four minutes for a more direct comparison to FEF catheters. These parameters allowed a comparison of FEF catheters to irrigated tip RF catheters under optimal conditions. The FEF catheters are not irrigated and lesions were applied to similar tissue slices in the same circulating bath under similar conditions. Contact force, tip temperature, starting impedance, and impedance drop were continuously monitored during each ablation lesion.

Infrared Thermography and Optical Imaging

Digital infrared thermal imaging (ITI) videos were recorded during RF applications using a 3rd generation infrared thermal camera (Shot PRO, Seek Thermal, Santa Barbara, Calif.). The infrared camera's image resolution was 320×240 (76,800 pixels) at a frame rate of <9 Hz. The thermal resolution of the camera was <0.07° C. with spectral range of 7.5-14 microns and maximum detectable temperature range of −40° C. to 330° C. The infrared camera's temperature range was manually set to 0° C. to 120° C. during experiments. The camera's emissivity was set to 0.90 based on previous estimates of emissivity in biologic tissue.

For each lesion, ITI video recordings were reviewed and still images were acquired at 30 second intervals (time: 0 sec, 30 secs, 60 secs, 90 secs, 120 secs, 150 secs, 180 secs, 210 secs, and 240 secs). Using proprietary software (Seek Fusion™, Seek Thermal, Santa Barbara, Calif.) ITI images were electronically registered and merged with optical images taken concurrently by the infrared thermal camera. Areas circumscribed by 100° C. Isotherms (mm²) were measured using commercially available image analysis software (Digimizer, MedCalc Software Ltd. Belgium) from video stills taken at 30 second intervals starting from onset to end of RF delivery. A ruler placed nearby the lesion set during image acquisition was used for calibration of measurements. In event of steam pops, RF delivery was continued unless the catheter tip was dislodged from the myocardial tissue interface during lesion application.

The still image for each lesion at 90 seconds was used to compare the temperature variation between catheters as a function of depth. The temperature was measured at 2 mm increments and averaged data used to create a plot comparing the temperature variation during lesion formation.

Tissue Staining and Analysis

Immediately following RF delivery, the myocardial samples were sliced in cross-section through the center of each lesion. One of the sliced sides of the sample was submerged and stained for 4 hours in 2% triphenyl tetrazolium chloride (TTC) solution to aid in differentiating the border of non-viable myocardial tissue of the lesion from healthy myocardium which stains red due to preservation of dehydrogenase enzymes. Optical photographs were obtained following TTC staining. Measurements of lesion diameter and depth at maximum lesion diameter were made using a digital micrometer.

Statistical Analysis and Results

Continuous variables are expressed as mean±standard deviation if normally distributed or otherwise as median (interquartile range [IQR]; 25th-75^{th} percentile). A Student t-test or Mann-Whitney U-test was used for parametric and nonparametric continuous variables, respectively. SPSS software was used to perform all calculations (Version 25, IBM, Chicago, Ill.). A p value of <0.05 was considered statistically significant.

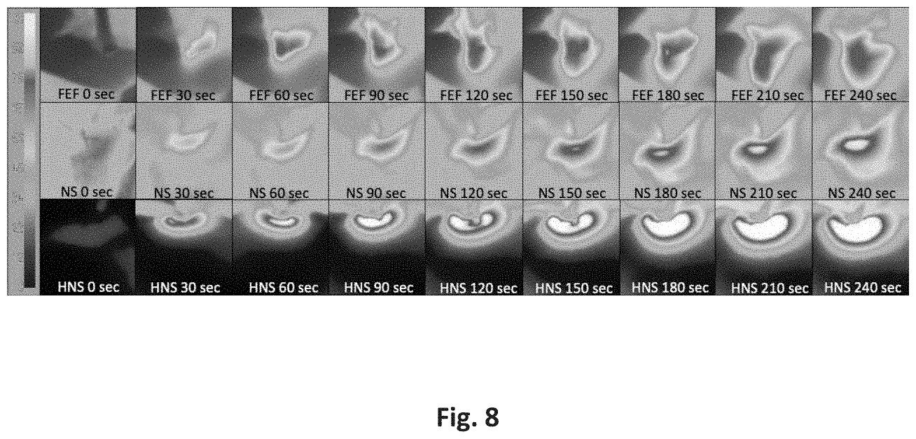

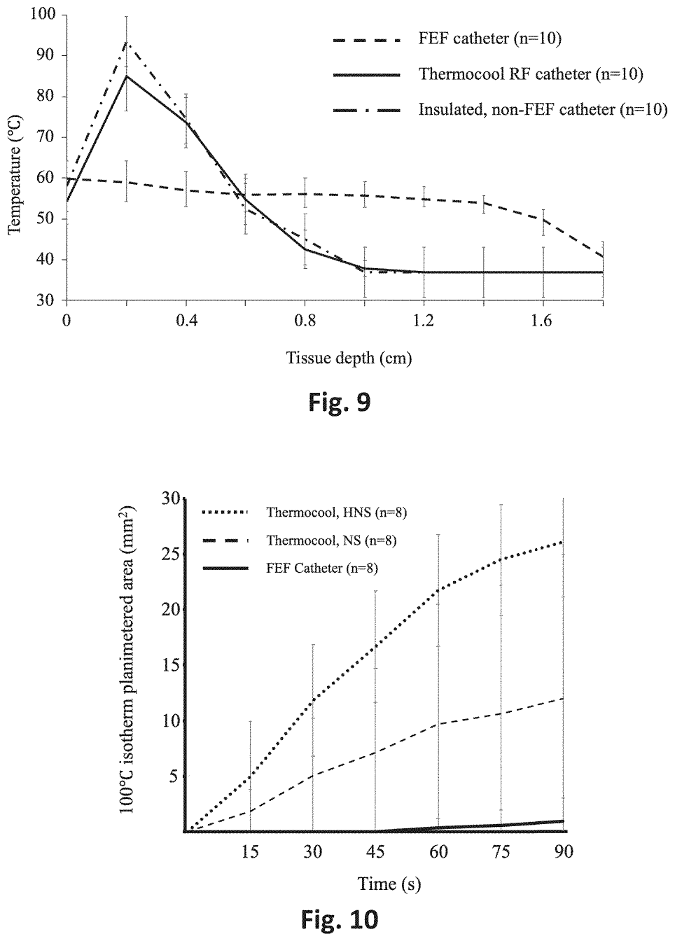

Results show that FEF ablation technology creates a different electric field configuration than conventional RF catheters. FIG. 7 shows RF lesions using TTC stained tissue sections post ablation comparing Thermocool and FEF catheter lesions. As seen visually and summarized in Table 1, FEF lesions are significantly deeper than RF lesions. FIG. 8 shows real-time thermal imaging comparing development of FEF lesions with Thermocool using normal saline (NS) and half-normal saline (HNS) lesions. FIG. 9 is a graph summarizing the variation of temperature with depth from the catheter-tissue interface. The addition of the concave FEF ablating surface leads to a markedly different temperature distribution. FIG. 10—is a graph showing planimetered area of 100° C. isotherm during ablation with FEF, HNS and NS ablation over time as a surrogate for the relative volume of tissue heated to the point of steam pop formation.

As described herein, ex vivo data demonstrates the superior ability of FEF ablation to target deeper tissues than conventional RF catheter technology. FEF lesions delivered at 20 W over 4 minutes were markedly deeper and wider than RF lesions (15.2±1.1 mm deep×16.1±2 mm wide). Importantly the incidence of steam pops was lower with FEF than with the Thermocool catheter using our setup. With RF ablation an incidence of 15% of steam pops was observed, comparable to other reports in ex vivo tissue. The incidence of steam pops was significantly lower with the FEF catheter, with pops occurring on 6.7% (2 out of 30) of lesions.

In these examples the control catheter was used under similar conditions as the FEF catheter. Using a power-controlled mode ablation above 20 W was found to invariably led to steam pops, so experiments were limited to 20 W. Due to the smaller area exposed to the tissue the starting impedance was higher for this catheter (175.7±28.0 vs 124.9±5.2 ohms for the FEF catheter). Impedance drop was similar for both (49.6±22.2 ohms for the insulated catheter vs 45.5±5.1 ohms for the FEF catheter). Lesions were 9.8±1.1 mm wide by 6.9±0.7 mm deep (n=10 experiments), similar to the Thermocool catheter and much smaller than the FEF catheter (15.2±1.1 mm deep×16.1±2 mm wide). These results are summarized in Table 1.

TABLE 1

| | | | | | Power | Steam |
|---|---|---|---|---|---|---|
| Catheter | Width (mm) | Depth (mm) | Starting ohms | Δ ohms | (W) | Pops |
| FEF: 2 min (n = 20) | 9.1 ± 1.7 | 11.8 ± 1.7 | 122.7 ± 5.7 | 32.7 ± 5.9 | 20 | 1 |
| FEF: 4 min (n = 10) | 16.1 ± 2.0 | 15.2 ± 1.1 | 124.9 ± 5.2 | 45.5 ± 5.1 | 20 | 1 |
| Insulated, non-FEF catheter (n = 10) | 6.9 ± 0.75 | 9.8 ± 1.1 | 175.7 ± 28.0 | 49.6 ± 22.2 | 20 | 8 |
| 40 W Thermocool: 90 s (n = 20) | 10.4 ± 1.2 | 6.2 ± 1.5 | 120 ± 4.3 | 18.2 ± 8.3 | 40 | 6 |
| 40 W Thermocool HNS: 90 s (n = 20) | 11.1 ± 1.0 | 6.9 ± 1.1 | 122.9 ± 8.7 | 21.3 ± 8.7 | 40 | 9 |
| 30 W Thermocool NS, 4 min (n = 6) | 13.8 ± 1.1 mm | 8.4 mm ± 1 mm | 134.8 ± 10.6 | 26.0 ± 9.9 | 20 | 5 |

Further data was collected using an infrared thermal camera. FIG. 8 compares the time course of lesion formation using the FEF catheter with a Thermocool catheter using either NS or HNS irrigation. RF ablation creates high tissue temperatures at the catheter-tissue interface with rapid fall-off with distance from the catheter. This is consistent with the known biophysics of RF ablation; RF lesions are created by superficial heating of the tissue by high current density near the catheter tip that then falls off rapidly with distance, and most lesion spread occurs by conductive heating. In contrast, with FEF ablation the hottest tissue temperature is located approximately 4-5 mm in depth, with a much more uniform temperature profile.

These data are summarized in FIG. 9, a plot of the tissue temperature at various depths recorded at the end of either an FEF, RF ablation or control catheters. With the Thermocool catheter, the temperature rises to a maximum of 85° C. at 2 mm depth. From this maximum the temperature falls off quickly, falling below 50° C. at 6 mm. The relatively cooler surface temperature is a consequence of surface cooling due to irrigation. The FEF lesion, in contrast, displays a different temperature distribution. The temperature never rises above 60° C. and does not fall below 50° C. until a depth of 1.6 cm. Both experimental and theoretical data demonstrate that FEF ablation relies on an electric field geometry with a slower fall-off with tissue depth that leads to ablation in a deeper zone of resistive heating. Insulating the sides of the RF catheter had no effect on the temperature distribution as shown in FIG. 9), confirming the role of the truncated catheter tip in forming deep ablation lesions.

The risk of steam pops was lower with FEF ablation due to more uniform tissue heating. As discussed above, steam pops occur when localized tissue heating leads to boiling of water within the tissue. Despite deeper ablation lesions, the incidence of steam pops was much lower with FEF ablation (2 steam pops out of 30 lesions for FEF vs 15 out of 40 lesions with the Thermocool catheter vs. 8 out of 10 with insulated solid-tip control catheter), as summarized in Table 1. To investigate the safety of FEF ablation and mechanism by which steam pops are eliminated, the amount of tissue that was heated to 100° C. during ablations using FEF and Thermocool catheters were compared with NS and HNS irrigation. The tissue reaching 100° C. during lesions is readily identified in thermal imaging sections, and the amount of tissue reaching this temperature using 2-dimensional planimetry of the 100° C. isotherm as a surrogate for the total tissue volume heated to this temperature were compared.

FIG. 10 summarizes the results of these experimental examples. Using NS as irrigant with a Thermocool catheter, a small core of tissue is heated to 100° C. by 90 seconds, consistent with our data in FIG. 9; this volume of tissue however continues to expand significantly over time with longer lesions. Using HNS ablation, the core of 100° C. tissue forms sooner and is much larger than with NS lesions (26 mm² planimetered area for HNS lesion at 4 minutes vs 10 mm² for the NS irrigant), which may explain recent reports in literature of increased steam pop rate with HNS ablation. Comparing the two RF curves with the FEF ablation curve demonstrates the markedly smaller extent of tissue heating near 100° C. using the FEF catheter compared to RF ablation with both NS and HNS irrigant, even while creating significantly deeper lesions (see also FIG. 10 for direct visual comparison of the volume of 100° C. tissue using the three ablation techniques).

These examples show that in an ex vivo porcine heart model the FEF catheter tip achieves much deeper ablation lesions than conventional RF catheters using lower power and causing fewer steam pops. Indeed, in a direct comparison with conventional catheters, FEF can reproducibly achieve over double the lesion depth. Steam pop incidence was shown to be significantly lessened, which in use would lower the risk of potentially life-threatening perforation of the heart and tamponade. Further, the control insulated catheter with a planar end was unable to achieve a similar temperature distribution or depth of lesions as the FEF catheter constructed in accord with embodiments of the instant disclosure. The results of these examples were validated using real-time infrared thermal imagery, showing that RF ablation with conventional catheters generate the greatest increase in temperature near the catheter-tissue interface where the electric field is highest. There is shown to be a rapid fall off in the amount of delivered electric energy with distance from the catheter tip, and most of the lesion volume is created by thermal conduction. Attempts at increasing power with standard RF catheters will inevitably be limited by hyperthermia of these more superficial tissue layers with risk of steam pop. The planimetered data discussed above demonstrate a significantly higher volume of tissue that reaches 100° C. with standard RF ablation than FEF. Without being bound by theory, higher energy delivery to tissue is likely the mechanism for the larger volume of tissue heated to 100° C. using half-normal saline irrigant, and consistent with recent reports suggesting more steam pops with half-normal saline than normal saline. In contrast, FEF ablation achieves more uniform and deeper heating of the tissue, with more of the lesion created by resistive than conductive heating.

These examples show that FEF technology using a novel catheter tip geometry achieves more uniform and deep lesions through a collimative effect on the electric field. The data reported herein suggests that FEF technology can create deeper ablation lesions than conventional RF ablation catheter technology with an improved safety margin. FEF technology can be used to target arrhythmic foci located at sites that are challenging to ablate with conventional technology, such as the LV summit, interventricular septum, and papillary muscles as well as other intramural and intramyocardial sites.

The Examples disclosed herein using an illustrative embodiment of the FEF catheter tip design demonstrate that the electric field remains confined to a narrow tissue region thus avoiding the rapid fall-off in energy delivery from the tissue surface that is inherent to conventional RF technology. FEF ablation according to the current disclosure advantageously allows significantly deeper ablation than current RF catheter technologies with an improved safety margin. The catheter is designed for use with both RF energy from standard generators as well as with PFA.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. An ablation catheter for ablating cardiac tissue comprising:
a catheter body having a longitudinal axis, the catheter body including a distal tip;
an ablation electrode proximate to the distal tip of the catheter body;
wherein the electrode has at least a partial dome shape which is at least partially electrically conductive.

2. The ablation catheter of claim 1 wherein the electrode further comprises a ceiling region which is non-conductive.

3. The ablation catheter of claim 1 wherein the dome shape of the electrode has a central axis which is parallel to the longitudinal axis of the catheter body.

4. The ablation catheter of claim 1 wherein the dome shape of the electrode has a central axis which is perpendicular to the longitudinal axis of the catheter body.

5. The ablation catheter of claim 1 comprising a plurality of electrodes spaced about the catheter body.

6. The ablation catheter of claim 5 wherein each of the plurality of electrodes has a central axis.

7. The ablation catheter of claim 6 wherein one or more of the central axes are parallel to one another or be at an angle to each other.

8. The ablation catheter of claim 6 wherein one or more of the central axes are non-parallel to one another.

9. The ablation catheter of claim 1 further comprising a temperature sensor in proximity to the electrode.

10. The ablation catheter of claim 1 further comprising at least one fluid aperture.

11. The ablation catheter of claim 10 having a fluid flow inlet aperture and a separate fluid flow outlet aperture.

12. The ablation catheter of claim 1 wherein the electrode is made of conductive mesh.

13. The ablation catheter of claim 1 wherein the electrode is made of conductive foldable mesh.

14. The catheter electrode of claim 1 wherein the dome shape of the electrode comprises a rim, and wherein the rim is covered by an insulating material.

15. An ablation catheter system for ablating cardiac tissue comprising:
an ablation catheter having:
a catheter body having a longitudinal axis, the catheter body including a distal tip;
at least one ablation electrode proximate to the distal tip of the catheter body;
wherein the electrode has at least a partial dome shape which is at least partially electrically conductive; and
an RF or PFA power generator connectable to the ablation catheter.

16. The ablation catheter system of claim 15 wherein the power generator is configurable to deliver RF or PFA power with time-dependent amplitude.

17. A method for cardiac ablation comprising the steps of:
a) providing an ablation catheter comprising:
a catheter body having a longitudinal axis, the catheter body including a distal tip;
at least one ablation electrode proximate to the distal tip of the catheter body;
wherein the electrode has at least a partial dome shape which is at least partially electrically conductive;
b) advancing the catheter tip to a cardiac treatment site of a patient;
c) delivering RF power to the electrode of the ablation catheter to ablate cardiac tissue.

18. The method of claim 17 wherein the cardiac tissue proximate to the tip of the catheter during the delivering step is heated less than compared to an electrode not having an at least a partial dome shape which is at least partially electrically conductive.

19. The method of claim 17 wherein the cardiac tissue proximate to the tip of the catheter during the delivering step exhibits fewer steam pops than compared to an electrode not having an at least a partial dome shape which is at least partially electrically conductive.

20. The method of claim 17 wherein the cardiac tissue ablated is located greater than 0.6 cm from the catheter tip and is ablated more quickly than compared to an electrode not having an at least a partial dome shape which is at least partially electrically conductive.
